(19) United States
(12) Patent Application Publication
(10) Pub. No.: US 2006/0088858 A1
(43) Pub. Date: Apr. 27, 2006

(54) Modified venom and venom components as anti-retroviral agents

(76) Inventors: Paul F. Reid, Plantation, FL (US); Laurence N. Raymond, Plantation, FL (US)

Correspondence Address: Robert J. Van Der Wall;Gables One Tower, Suite #1275, 1320 South Dixie Highway, Coral Gables, FL, 33146, US

(21) Appl. No.: 11/217,713

(22) Filed: Sep. 2, 2005

Related U.S. Application Data
(63) Continuation-in-part of application No. 10/883,834, filed on Oct. 25, 2004.

(51) Int. Cl. C12Q 1/70; C12Q 1/68

(52) U.S. Cl. 435/6; 435/5

(57) ABSTRACT

The present invention relates to a class of proteins, and a method for treatment of neurological and viral diseases in humans and animals. More specifically it applies to the treatment of heretofore intractable diseases such as retro-viral infections including human immunodeficiency virus (HIV), simian immunodeficiency virus (SIV), feline immunodeficiency virus (FIV), bovine immunodeficiency virus (BIV) and equine acquired immunodeficiency virus (EAIV). The method of treatment comprises administering to the subject a disease mitigating amount of a detoxified modified venom composition.

### BACKGROUND OF THE INVENTION

1. Field of the Invention

The present invention relates to a method for treatment of retro-viral infections, including specifically HIV infections.

2. Description of Prior Art

Sanders, et al. had commenced investigating the application of modified venoms to the treatment of ALS in 1953 having employed poliomyelitis infection in monkeys as a model. Others antiviral studies had reported inhibition of pseudorabies (a herpesvirus) and Semliki Forest virus (alpha-virus). See Sanders' U.S. Pat. Nos. 3,888,977, 4,126,676, and 4,162,303. Sanders justified the pursuit of this line of research through reference to the studies of Lamb and Hunter (1904) though it is believed that the original idea was postulated by Haast. See Haast U.S. Pat. Nos. 4,341,762 and 4,741,902. See also MacDonald, et al., U.S. Pat. No. 5,723,477. The studies of Lamb and Hunter (Lancet 1:20, 1904) showed by histopathologic experiments with primates killed by neurotoxic Indian cobra venom that essentially all of the motor nerve cells in the central nervous system were involved by this venom. A basis of Sanders' invention was the discovery that such neurotropic snake venom, in an essentially non-toxic state, also could reach that same broad spectrum of motor nerve cells and block or interfere with invading pathogenic bacteria, viruses or proteins with potentially deleterious functions. Thus, the snake venom used in producing the composition was a neurotoxic venom, i.e., causing death through neuromuscular blockade. As the dosages of venom required to block the nerve cell receptors would have been far more than sufficient to quickly kill the patient, it was imperative that the venom was detoxified. The detoxified but undenatured venom was referred to as being neurotropic. The venom was preferably detoxified in the mildest and most gentle manner. While various detoxification procedures were known then to the art, such as treatment with formaldehyde, fluorescein dyes, ultraviolet light, ozone, heat, it was preferred that gentle oxygenation at relatively low temperatures be practiced, although the particular detoxification procedure was not defined as critical. Sanders employed a modified Boquet detoxification procedure using hydrogen peroxide, outlined below. The acceptability of any particular detoxification procedure was tested by the classical Semliki Forest virus test, as taught by Sanders, U.S. Pat. No. 4,162,303.

U.S. Pat. No. 3,888,977, issued on Jun. 10, 1975 to Murray J. Sanders (the entire disclosure of which is incorporated herein by reference and relied upon for details of disclosure) teaches that animals, including humans, may be treated for progressive degenerative neurological diseases, such as amyotropic lateral sclerosis, by administration of a modified snake venom neurotoxin derived from the venom of either the Bungarus genus, Naja genus or from a combination of the Bungarus genus and the Naja genus, i.e., in either case the therapeutic composition must contain at least in part modified neurotoxin derived from the Bungarus genus. Thus, it is taught that while the Bungarus venom can be effectively used alone, the Naja venom must be used in combination with the Bungarus venom. Unfortunately, however, Bungarus venom is not as readily available as Naja venom; the supply thereof is more uncertain; and it is far more expensive than the Naja venom. Sanders U.S. Pat. No. 4,126,676 (1978) provided a method of treatment of animals suffering from progressive degenerative neurological diseases wherein the therapeutic modified neurotoxin was derived from the Naja genus alone. Miller, et al. (1977) reported that the modified venoms antiviral activity against Semliki Forest virus was associated with several chromatographic fractions comprising the neurotoxic components. The most abundant component with antiviral activity was shown to be alpha-cobratoxin. Yourist, et al. (1983) reported that modified alpha-cobratoxin could inhibit the activity of herpesvirus. It seemed therefore, that these modified venoms and constituents had significant inhibitory activity against unrelated viruses. This non-specific activity has prompted the examination of these modified venom products against a number of viral types.

Other references of interest include four patents, Haast, U.S. Pat. Nos. 4,741,902 and 5,723,477, Hoxie, U.S. Pat. No. 5,994,515 and Au-Yuong, et al., U.S. Pat. No. 5,955,303. Literature references of interest are: Battaglioli E., Gotti C., Terzano S., Flora A., Clementi F. and Formasari D.; J. Neurochem. 71:1261-1270 (1998), Benhammou K., Lee M., Strook M., Sullivan B., Logel J., Raschen K., Gotti C. and Leonard S.; Neuro-pharmacology 39:2818-2829 (2000), Bewley C. A. and Otero-Quintero S.; J. Am. Chem. Soc. 123:3892-3902 (2001), Boquet P.; Ann. Inst. Pasteur 66:379-396 (1941), Boyd M. R., Gustafson K. R., McMahon J. B., Shoemaker R. H., O'Keefe B. R., Mori T., Gulakowski R. J., Brockes J. P. and Hall Z. W.; Biochemistry 14:2092-2099 (1975), Boyle M. J., Conners M., Flanigan M. E., Geiger S. P., Ford H. Jr., Baseler M., Adelsberger J., Davey R. T. Jr., Lane H. C.; J. Immunol. 154:6612-6623 (1995), Bhattacharya T.; Science 288:1789-1796 (2000), Bonyhadi M. L., Su L., Auten J., McCune, J. M., Kaneshima, H.; AIDS Res. & Hum. Retroviruses 11:1073-1080 (1995), Bracci L., Lozzi L., Rustici M. and Neri P.; FEBS 311:115-118 (1992), Bracci L., Ballas S. K., Spreafico A. and Neri P.; Blood 90:3623-3628 (1997), Cammack N.; Curr. Opin. Infect. Dis. 14:13-16 (2001), Chang L. C. and Bewley C. A.; J. Mol. Biol.:318:1-8 (2002), Chang C. C., Kawata Y., Sakiyama F. and Hayashi K.; Eur. J. Biochem. 193:L567-572 (1990), Choe H., Farzan M., Sun Y., Sullivan N., Rollins B., Ponath P. D., Wu L., Mackay C. R., LaRosa G., Newman W., Gerard N., Gerard C. and Sodroski J.; Cell 85:1135-1148 (1996), Collins K. B., Patterson B. K., Naus G. J., Landers D. V., Gupta P.; Nature Med. 6:475-479 (2000), Courgnaud V., Pourrut X., Bibollet-Ruche F., Mpoudi-Ngole E., Bourgeois A., Delaporte E. and Peeters M.; J. Virol. 75:857-866 (2001), Davies B. D., Hoss W., Lin J. P. and Lionetti F.; Mol. Cell Biochem. 44:23-31 (1982), Derdeyn C. A., Decker J. M., Sfakianos J. N., Wu X., O'Brien W. A., Ratner L, Kappes J. C., Shaw G. M., and Hunter E; J. Virol. 74:8358-8367 (2000), De Clerque E.; Mini. Rev. Med. Chem. 2:163-175 (2002), Deng H., Liu R., Ellmeier W., Choe S., Unutmaz D., Burkhart M., diMarzio P., Marmaon S., Sutton R. E., Hill C. M., Davis C. B., Peiper S. C., Schall T. J., Littman D. R. and Landau N. R.; Nature 381:661-666 (1996), Dowding A. J. and Hall Z. W.; Biochemistry 26:6372-6381 (1987), D'Souza M. P., Cairns J. S. and Plaeger S. F.; J.A.M.A. 284:215-222 (2000), Esser M. T., Mori T., Mondor I, Sattentau Q. J., Dey B, Berger E. A., Boyd M. R. and Lifson J. D.; J. Virol. 73:4360-4371(1999), Feng Y., Broder C. C., Kennedy P. E. and Berger E. A.; Science 272:872-877 (1996), Franti M., O'Neill, Maddon P., Burton D. R., Poignard P. and Olson W.; 9^{th} Conference on retroviruses and opportunistic infections; February 24-28, Washington State Convention Trade Center, Seattle Wash.; 2002, Froehner S. C. and Rafto S.; Biochemistry 18:301-307 (1979), Fujii T., Tsuchiya T., Yamada S., Fujimoto K., Suzuki T., Kasahara T. and Kawashima K.; J. Neurosci. Res. 44:66-72 (1996), Fujii T. and Kawashima K.; Jpn. J. Pharmacol. 85:11-15 (2001), Greenhead P., Hayes P., Watts P. S., Laing K. G., Griffin G. E. and Shattock R. J.; J. Virol. 74:5577-5586 (2000), Gustafson K. R., Sowder R. C., Henderson L. F., Cardellina J. H., McMahon J. B., Rajamani U., Pannell L. K. and Boyd M. R.; Biochem. Biophys. Res. Commun. 238:223-228 (1997), Grabczewska E., Laskowska-Bozek H., Maslinski W. and Ryzewski J.; Int. J. Tissue React. 12:281-289 (1990), Hallquist N., Hakki A., Wecker L., Friedman H. and Pross S.; Proc. Soc. Exp. Biol. Med. 224:141-146 (2000), Hanna S. L., Yang C., Owen S. M. and Lal. R. B.; AIDS 16:1603-1608 (2002), Hiemke C., Stolp M., Reuss S., Wevers A., Reinhardt S., Maelicke A., Schlegel S. and Schroder H.; Neurosci. Lett. 214:171-174 (1996), Horn T. and Braun J. F.; P.R.N. Notebook: http://www.prn.org_nb_cntnt/cap08-15-02.06.htm, Jiang S., Zhao Q. and Debnath A. K.; Curr. Pharm. Des. 8:563-580 (2002), Kawashima K. and Fujii T.; Pharmacol. Ther. 86:29-48 (2000), Kawashima K., Fujii T., Watanabe Y. and Misawa H.; Life Sci. 62:1701-1705 (1998), Kilby J. M., Hopkins S., Venetta T. M., DiMassimo B., Cloud G. A., Lee J. Y., Alldredge L., Hunter E., Lambert D., Bolognesi D., Matthews T., Johnson M. R., Nowak M. A., Shaw G. M. and Saag M. S.; Nat. Med. 4:1302-1307 (1998), Kolchinsky P., Kiprilov E., Bartley P., Rubinstein R. and Sodroski J.; J. Virol. 75:3435-3443 (2001), Korber B., Muldoon M., Theiler J., Gao F., Gupta R., Lapedes A., Hahn B. H., Wolinsky S., Bhattacharya T.; Science 288:1787-1796 (2000), Koyanagi Y., Tanaka Y., Kira J., Ito M., Hioki K., et al.; J. Virology 71:2417-2424 (1997), Lamb G. and Hunter, W. K., Lancet, 1: 20-22 (1904), Lentz T. L., Burrage T. G., Smith A. L., Crick J. and Tigor G. H.; Science 215:182-184 (1982), Lenz T. L., Hawrot E. and Wilson P. T.; Proteins:Structure, Function and Genetics 2:298-307 (1987), Levin J.; Report; 42^{nd} ICAAC Meeting, San Diego, Sept. 27-31 (2002); Mariner J. M., McMahon J. B., O'Keefe B. R., Nagashima K. and Boyd M. R.; Biochem. Biophys. Res. Commun. 30:841-845 (1998), Markham R. B., Schwartz D. H., Templeton A., Margolick J. B., Farzadegan H., et al.; J. Virology 70:6947-6954 (1996), McLane K. E., Fritzen M., Wu X., Diethelm B., Maelicke A. and Conti-Tronconi B. M; J. Recept. Res. 12:299-321 (1992), McLeod G. X., McGrath J. M., Ladd E. A., Hammer, S. M.; Antimicrob. Agents Chemother. 36:920-925 (1992), Miller K. D., Miller G. G. and Sanders M., Fellows O. N.; Biochem. Biophys. Acta 496:192-196 (1977), Mizuno Y., Dosch H. M. and Gelgand E. W.; J. Clin. Immunol. 2:303-308 (1982), Moore J. P., Sattentau Q. J., Wyatt R. and Sodroski J.; J. Virol. 68:469-484 (1994), Mori T. and Boyd M. R; Antimicro. Agents Chemother. 45:664-672 (2001), Myers G. and Lu H.; http://hiv-web.lanl.gov/content/hiv-db/REVIEWS/articles Nagashima K. A., Thompson D. A., Rosenfield S. I., Maddon P. J., Dragic T. and Olson W. C.; J. Infect. Dis. 183:1121-1125 (2001), Neri P., Bracci L., Rustici M. and Santucci A.; Arch. Virol. 114:265-269 (1990), Patterson, B., Flener, Z., Yogev, R. and Kabat, W. “Inhibition of HIV-1 replication in mononuclear cells and thymus explant cultures by a purified, detoxified cobra venom protein” (2000) Abstract, “Novel biological fusion inhibitors of HIV”, Apr. 7, 2000, Keystone Conference, Colorado, Peters B. S.; Antivir. Chem. Chemother. 11:311-320 (2000), Piot P.; Science 280:1844-1845 (1998) Reeves J., Puffer B., Ahmad N., Derdeyn C., Sharron M., Edwards T., Carlin D., Harvey P., Pierson T., Hunter E. and Doms R. W.; 9^{th} Conference on retroviruses and opportunistic infections; Feb. 24-28, Washington State Convention Trade Center, Seattle Wash.; 2002, Rusconi S., Moonis M., Merrill D., Pallai P. V., Neidhardt E. A., Singh S. K., Willis K. J., Osburne M. S., Profy A. T., Jenson J. C. and Hirsch M. S.; Antimicrobial Agents and Chemotherapy 40:234-236 (1996), Schearer W. T., Israel R. J., Starr S., Fletcher C. V., Wara D., Rathore M., Church J., DeVille J., Fenton T., Graham B., Samson P., Staprans S., McNamara J., Moye J., Maddon P. J. and Olson W. C.; J. Infect. Dis. 182:1774-1779 (2000), Sato K. Z., Fujii T., Watanabe Y., Yamada S., Ando T., Kazuko F. and Kawashima K.; Neurosci. Lett. 26617-20 (1999), Sanders M. and Fellows O.; (1974) In Excerpta Medica; International Congress Series No. 334 containing abstracts of papers presented at the III International Congress of Muscle Diseases, Newcastle on Tyne, September, Sanders, M., Soret, M. G. and Akin, B. A.; Ann. N.Y. Acad. Sci. 53: 1-12 (1953), Sanders, M., Soret, M. G., and Akin, B. A.; J. Path. Bacteriol. 68:267-271 (1954), Sanders, M., Soret M. G. and Akin B. A.; J. Path. Bact. 68:267-271 (1954a), Sanders, M., Soret M. G. and Akin B. A.; Acta Neurovegetat 8:326-327 (1954b) Sanders, M., Soret M. G. and Akin B. A., Roizin L.; Science 127:594-596 (1958a), Sanders, M., Soret M. G. and Akin B. A.; Proc 7^{th} Inter. Cong. Microbiol.; p. 293 (1958b), Sanders, M. and Fellows O.; Cancer Cytology 15:34-40(1975) Schols D., Este J. A., Henson G. and Declerq E.; Antiviral Res. 35:147-156 (1997), Schols D., Claes S., De Clercq E., Hendrix C., Bridger G., Calandra G., Henson G., Fransen S., Huang W., Whitcomb J. M. and Petropoulos J; 9^{th} Conference of retroviruses and opportunistic infections; (2002), Singh S. P., Karla R., Puttfarcken P., Kozak A., Tesfaigzi J. and Sopori; Toxicol. Appl. Pharmacol. 164:65-72 (2000), Starcich B. R., Hahn B. H., Shaw G. M., McNeely P. D., Modrow S, Wolf H., Parks E. S., Parks W. P., Josephs S. F. and Gallo R. C.,; Cell 45:637-648 (1986), Sullivan N., Sun Y., Sattentau Q., Thali M., Wu D., Denisova G., Gershoni J., Robinson J., Moore J., and Sodroski J.; J. Virol. 72:4694-4703 (1998), Thali M., Moore J. P., Furman C., Charles M., Ho C. C., Robinson J. and Sodroski J.; J. Virol. 67:3978-3988 (1993), Toyabe S., Iiai T., Fukuda M., Kawamura T., Suzuki S., Uchiyama M. and Ado T.; Immunology 92:201-205 (1997), Tremblay C. L., Kollmann C., Giguel F., Chou T. C. and Hirsch M. S.; J. Acquir. Immune Defic. Syndr. 25:99-102 (2000), Tu A. T.; Ann. Rev. Biochem. 42:235-258(1973) VanDamme L., Wright A., Depraetere K., Rosenstein I., Vandermissen V., Poulter L., McKinlay M., Van Dyck E., Weber J., Profy A., Laga M. and Kitchen V.; Sex. Transm. Infect. 76:126-130 (2000), Weber J., Nunn A., O'Conner T., Jeffries D., Kitchen V., McCormack S., Stott J., Almond N., Stone A. and Darbyshire J.; AIDS 15:1563-1568 (2001), Wei X., Decker J. M., Liu H., Zhang Z., Arani R. B., Kilby J. M., Saag M. S., Wu X., Shaw G. M. and Kappes J. C.; Antimicrob. Agents Chemother. 46:1896-1905 (2002), Wild C. T., Shugars D. C., Greenwell T. K., McDanal C. B. and Matthews T. J.; Proc. Natl. Acad. Sci. 91:9770-9774 (1994), Wu L., Gerard N. P., Wyatt R., Choe H., Parolin C., Ruffing N., Borsetti A., Cardoso A. A., Desardin E., Newman W. and Sodroski J.; Nature 384:179-183(1996), Wu L., Rivera M. I., Laurencot C. M., Currens M. J., Cardellina J. H., Buckheit R. W., Nara P. L., Pannell L. K., Sowder R. C. and Henderson L. E.; Antimicro. Agents and Chemotherapy 41:1521-1530 (1997), Wyatt R., Moore J., Accola M., Desjardin E., Robinson J. and Sodroski J.; J. Virol. 69:5723-5733 (1995), Zhu C. B., Zhu L., Holz-Smith S., Matthews T. J. and Chen C. H.; PNAS 98:15227-15232 (2001).

### SUMMARY OF THE INVENTION

The present invention provides a composition and method for treating and preventing retroviral infections of mammalian cells. One aspect of the invention relates to an retroviral composition derived from modified venom which can be administered in-vivo for the treatment of HIV infection. In another aspect, the invention relates to the synergistic effects of modified venom constituents in preventing HIV infection and replication. In another aspect, the retrovirus is selected from the group consisting of Lentiviruses (HIV-1, HIV-2, SIV, EIAV, BIV, and FIV).

Proteins such as those from venoms, as described herein, have long been recognized for their ability to bind to specific receptors on the surface of mammalian cells. These neurospecific proteins bind to such common receptors as the acetylcholine receptor for example. However, the protein motif employed by these neurotoxins to affect binding appears to be a common motif employed by other, apparently unrelated, proteins including those present in viral coat proteins. Such viral proteins include rabies virus coat protein and gp120 from HIV. Prior studies had indicated that proteins with these motifs could interfere with the activity of the other. Sanders provided a method which permits the safe administration of venom proteins allowing the application of these laboratory observations to practical use. Therefore included in the invention is a method of treating a lentivirus infection in mammals and humans comprising administering to the host the modified venom.

### DETAILED DESCRIPTION OF THE INVENTION

Although the survival of individuals currently infected by the HIV virus is dramatically longer than it was 20 years ago, such survival is at the cost of a drug regime which is highly expensive, complicated, relegated to a fixed time and sequence schedule, has adverse physiological side effects and is, ultimately, too little too late. While the logical method to halt the spread of the disease is sexual abstinence, such method embodies so many facets of world society, that, realistically, the disease will remain uncontrollable until such a time as it can be controlled by methods which are inexpensive, have few side effects, and can be administered easily.

Prophylaxis, utilized before or after potential exposure, fulfills these requirements. Potential prevention/treatment could take many forms; three are: 1. The development of a vaccine that prevents infection; 2. Prevention of an initial infection or control of the spread of an initial infection that has not progressed to AIDS by a means other than a vaccine, or, 3. A resolution of the syndrome known as AIDS by the use of anti-retroviral agents. While vaccine production is ultimately the most efficacious of the three methods, due to the mutational idiosyncrasies of the virus, such development is not a likely or a probable immediate occurrence. Vaccine development attempts to date have failed to translate into man from animal test-models (Peters; 2000).

Medical research resources are currently being applied to the management, rather than the cure of a HIV infection. While the use of anti-retrovirals agents have improved the quality and length of life, they have disadvantages which include toxicity, development of drug resistance, persistence of latently infected cells resulting in viral rebound after prolonged treatment and, finally, high expense. The prevention and/or control of an infection prior to loss of immune capabilities associated with progression to AIDS is currently the most expedient and cost effective method. Currently, there are several approved drugs types that apply themselves to the control of an ongoing HIV infection. These drug types are, nucleoside reverse transcriptase inhibitors, non-nucleoside reverse transcriptase inhibitors and protease inhibitors. These are currently encompassed by highly aggressive anti-retroviral therapy (HAART). All these drug types are susceptible to loss of effectiveness due to genetic mutation of the HIV-1. Thus, the blockade of HIV infection or the control of the spread of HIV infection through the use of fusion or entry inhibitors appears to be the most logical method barring the availability of a vaccine. Such blocking substance, or substances, could be applied topically, as a cream or douche, and provide protection during coitus. The use of this mode of prevention has been suggested by others (Turpin, 2002) and is being implemented (Van Damme, et al., 2000). The utilization of a binding/entry inhibitor as a prophylactic that would block infection and maintain a period of protection in the genital tract could provide an effective measure which would reduce HIV-1 transmission (D'Sousa, et al., 2000). Topical administration would not be amenable to prevention of disease by blood transfer by more direct routes (such as needles). However, as an injectable, or by buccal administration, it could be applicable parenterally in the treatment of an HIV infection during early stages of exposure, or later, by providing control of HIV dissemination within the host.

Alternatively, drug activities which alter the virus and reduce its infectivity or alter its functional form upon release would supply a mechanism for infection control at the “other side” of the infection sequence.

HIV-1 is a lentivirus (lenti=slow {Latin}) of the family Retroviridae. The virus is enveloped, 80-130 nm in diameter and has an icosahedral capsid. As with other lentiviruses, HIV can infect terminally differentiated, non-dividing cells such as macrophages resident in tissue or brain (microglia) as well as cells of the T cell lineage, specifically CD4+ cells, known as T helper (T_{H}) cells. Lentiviruses have, through mutation, the capability to infect immune cells (macrophages; T_{H}-cells), the ability to avoid immune system eradication and, thus, tend to persist for the life of their host. The typical HIV infection progresses through three stages: initial, or acute, associated with high levels of viral replication and dissemination, a latent stage attributed to partial immune system control, which is followed by the third stage which encompasses the return of high levels of viral replication and progression to clinical disease states due to decreased immunocompetence, termed acquired immunodeficiency syndrome (AIDS). HIV is suggested to be derived from the simian immunodeficiency virus (SIV) (Courgnaud, et al., 2001) and first entered the human population between 1915 and 1941 (Korber, et al., 2000). Two HIVs are associated with human AIDS: HIV-1 and HIV-2. HIV-1 is distributed worldwide and is responsible for the current AIDS pandemic while HIV-2 is currently restricted to West Africa. Both are spread by the same routes, though HIV-2 may be less pathogenic.

Treatment of HIV infection currently encompasses two basic modalities: drug action at host intracellular targets (post entry) and drug interaction at viral extracellular targets (pre-entry). The latter are termed as binding/entry inhibitors. Extracellular targets are those associated with viral attachment, fusion and entry into the host cell. Intracellular targets are those associated with viral nucleic acid synthesis and processing and are termed as anti-retroviral drugs. There are currently 16 licensed antiretroviral drugs employed to combat HIV-1 infection (D'Souza, et al. 2000, aidsmeds.com, 2002a). Currently, there is a drug, T-20 (Trimeris), which is licensed as a binding/entry inhibitor. Within the context of this proposal, extracellular targets are of immediate importance, consequently, discussions of viral inhibition post-cell entry will be omitted.

Infection by HIV occurs following the introduction of the virus to the blood of the potential host. Virus-host cell interaction is mediated through the viral envelope glycoproteins gp120 and gp41 (gp160), which are assembled as trimers on the surface of the viral envelope, and their interactions with host cell surface receptors CD4, and CXCR4 or CCR5. U.S. Pat. No. 5,994,515 (Hoxie) describes the manner in which the human immunodeficiency viruses HIV-1 and HIV-2 and the closely related simian immunodeficiency viruses (SIV), all use the CD4 molecule as a receptor during infection though viruses like HIV and FIV can infect CD4 negative cells. The latter two host cell surface receptors are chemokine receptors and act as co-receptors along with CD4. Chemokines are a large family of low molecular weight, inducible, secreted, proinflammatory cytokines which are produced by various cell types. See, for instance, Au-Yuong, et al., U.S. Pat. No. 5,955,303. Chemokines have been divided into several subfamilies on the basis of the positions of their conserved cysteines. The CC family includes monocyte chemoattractant protein-1 (MCP-1), RANTES (regulated on activation, normal T cell-expressed and secreted), macrophage inflammatory proteins (MIP-1.alpha., MIP-1.beta.), andeotaxin. (Proost, P. (1996) Int. J. Clin. Lab. Res. 26: 211-223; Raport, C. J. (1996) J. Biol. Chem. 271: 17161-17166). The CXC family includes interleukin-8 (IL-8), growth regulatory gene, neutrophil-activating peptide-2, and platelet factor 4 (PF-4). Although IL-8 and PF-4 are both polymorphonuclear chemo-attractants, angiogenesis is stimulated by IL-8 and inhibited by PF-4. However, the macrophage tropic (CCR5) strain BaL, is not capable of infecting cells which co-express both CXCR4 and CD4. These results suggest that CXCR4 can serve as a co-factor for T-tropic, but not M-tropic, HIV-1 strains (Feng, et al., 1996, supra). Moreover, the finding that there is a change from M to T-tropic viruses over time in infected individuals correlates with disease progression suggests that the ability of the viral envelope to interact with CXCR4 represents an important feature in the pathogenesis of immunodeficiency and the development of full blown AIDS.

There are five variable regions and five conserved regions that compose gp120 (Starcich, et al., 1986; Wyatt, et al., 1995). Two variable loop regions, V1/V2 and V3, prior to initial viral interaction with the cell surface, are closely associated and block accessibility to a region associated with chemokine receptor binding. Binding of CD4, which occurs above these two variable regions, is dependent upon discontinuous elements in conserved regions 3 and 4 (C3 and C4)(Moore, et al., 1994). Amino acid changes in the V2 and V3 loop regions can alter both the membrane fusion process and HIV-1 tropism (Wyatt, et al., 1995).

Infection of susceptible cells occurs via three conformational stages involving HIV-1 gp120 (D'Sousa et al., 2000). In short, the interaction between HIV-1 and the host cell proceeds as follows: A segment of gp120 binds to CD4 on the host cell surface resulting in an initial conformational change of the V1/V2 and V3 regions of gp120. This change allows access to a portion of gp120, previously covered by the two variable regions, which binds with a co-receptor resident on the host cell. This gp120 conformational change involves movement of the V1/V2 loops away from the V3 loop (Thali, et al., 1993; Wyatt, et al., 1995, Sullivan, et al., 1998). Under normal circumstances, HIV-1 gp120 requires the presence of both the CD4 and a co-receptor to cause additional conformational changes resulting in exposure of gp41. The viral protein, gp41, is responsible for fusion and entry. The CD4 co-receptor is either CXCR4 or CCR5 and is determined by the tropism of the virus (Feng, et al., 1996; Doranz, et al., 1996; Deng, et al., 1996; Choe, et al., 1996; Wu, et al., 1996). The extracellular portion of gp41 contains two helical domains: HR1 and HR2 (or NHR and CHR; Jiang, et al., 2002). The tip of gp41 inserts into the host cell membrane and anchors the virus to the cell. The two helical domains of gp41, previously separated by a segment of gp120, bind together to form a 6-helix bundle that is a fusogenic structure (Jiang, 2002). The virus and cell surface are pulled together by this structure, allowing fusion of the virus envelope and host cellular membrane and insertion of viral genetic material. The co-receptor CCR5, whose natural ligands are the a chemokines RANTES, MIP-1-a, MIP-1-b and MDC, is employed by primary isolates of HIV-1 which are generally M (macrophage) tropic, and is found on T cells and macrophages. CXCR4, whose natural ligand is SDF-1a, is employed by late stage HIV-1 isolates and is employed by T (T cell)-tropic HIV-1. There is an in vivo switch in tropism during HIV infection (Wyatt and Sodroski, 1998).

Due to the complexity of the binding and penetration of HIV-1, the virus is, at least theoretically, vulnerable to either single or, more especially, multiple entry inhibitors. Therefore, there are several cellular sites and viral sites with which inhibitors could interact to halt the process: CD4, CXCR4, CCR5, gp120 and gp41. The substances currently under consideration generally have high cost in addition to limited production as well as low bio-availability and poor pharmacologic and toxicology profiles. Nineteen potential binding/entry inhibitors were listed in 2000 (D'Sousa, et al., 2000); work is still progressing and a glance at the current literature indicates new additions in the list. Gp41 inhibitors T-20 and T-1249 (Trimeris/Hoffman LaRoche) as well as PRO-542 (Progenics), PRO-2000 (Procept) and Cyanovirin (CV-N) all of which target virus/CD4 interaction and AMD-3100 (AnorMed), which interferes with HIV/CXCR4 interactions, are still viable candidates. These compounds are representative of, and provide an overview of, current thought in the area of inhibiting viral binding/entry (De Clercq, 2002).

The drug candidates listed above suggest that combinatorial efforts to prevent binding and entry is likely to become the norm, as opposed to the use of single drugs, as indicated by the synergistic combination of drugs with T-20. Additionally, the concept of disease prevention by the use of binding/entry inhibitors is established in the research and clinical communities. The use of PRO-2000 in a vaginal gel, coupled with the early results achieved, suggest that this is a potentially viable approach, especially given that this is associated with the most frequent mode of transmission (Greenhead, 2000). This topical approach is strengthened by the determination that HIV must transit the epithelial lining of the vagina wall to access infection susceptible cells, that epithelial cells are not subject to infection and they do not aid transport of the virus. In fact, the epithelial cells may act as a barrier to infection. The presence of PRO2000 was found to result in 97% reduction in HIV infection in an in-vitro cervical explant test system (Greenhead, 2000).

Molecular mimicry; alpha-neurotoxin/HIV gp120 sequence homology

Death by cobra envenomation is attributed to the interaction of basic polypeptides (cobra alpha-neurotoxins) that act post-synaptically and result in blockade of nerve transmission due to their affinity for the nicotinic acetylcholine receptor (nAchR). nAchRs are ligand-gated ion channels activated by the binding of acetylcholine (Ach). On muscle, the nAchR molecule is a pentamer composed of two alpha subunits, one beta, one gamma and one delta subunit. Ach binds to the alpha subunit, each nAchR complex having two acetylcholine binding sites (Dowding et al., 1987). Cobratoxin and other snake alpha-neurotoxins are curaremimetic since they mimic the actions of curare in that they are potent competitive inhibitors of Ach binding to the nAchR and blocking Ach activity.

The alpha-neurotoxins of Naja kaouthia (cobratoxin) and Bungarus multicinctus (bungarotoxin) have a sequence homology with HIV gp120 and rabies virus glycoprotein (RVG) as indicated below in Table I. This homology is located in a manner that it is accessible for the production and interaction with antibodies on both viruses. Like the homologous sequence on elapid toxins, the amino acid sequence present in rabies virus glycoprotein (RVG) and gp120 of HIV results in interaction with the nAchR. This interaction has been demonstrated by the binding of rabies virus (Lentz, et al., 1982, Lentz, et al., 1987) and HIV-1 gp120 (Bracci, et al., 1992). Both viral interactions were blocked by the use of lpha-bungarotoxin.

TABLE I
SEQUENCE HOMOLOGY OF HIV-1, RVG and SNAKE NEUROTOXINS

| | | | | | | | | | | | | | | |
|---|---|---|---|---|---|---|---|---|---|---|---|---|---|---|
| RVG (189-199) | | C | D | I | F | T | N | S | R | G | K | R | | |
| HIV-gp120 (159-169) | | F | N | I | S | T | S | I | R | G | K | V | | |
| Peptide B2 | S | F | N | I | S | T | S | I | R | G | K | V | Q | K |
| cobratoxin (30-40) | | C | D | A | F | C | S | I | R | G | K | R | | |
| bungarotoxin (30-40) | | C | D | A | F | C | S | S | R | G | K | V | | |

From Neri et. al.; 1990; Bracci et. al.; 1992; Bracci et. al.; 1997, Meyers and Lu; 2002

In studies to confirm the reported anti-HIV activity of modified alpha-cobratoxin it was decided to include modified N. kaouthia venom as a comparator as this venom is the principal source of cobratoxin. Cobratoxin represents 15-20% of the venom composition and it was logical to assume that the modified venom product would have one fifth the activity of modified cobratoxin in antiviral assays. Naja naja venom was erroneously employed (in place of N. kaouthia) initially as a control for comparison. Several formulations of the modified N. naja venom demonstrated strong inhibition of the virus. However, the modified venom preparation unexpectedly demonstrated a higher inhibitory activity than that of the purified neurotoxin though the venom sample contained less than 20% of the expected active component (FIG. 1, table 1). It was then discovered that the venom material being tested was not N. kaouthia but N. naja. The appropriate inclusion of N. kaouthia in the HIV assay proved it to be a poor inhibitor of HIV replication and the dose response was more readily observed. The higher activity of N. naja venom suggested that cobratoxin was in fact not the sole active component against HIV and that there were other components contributing to the antiviral effect that were synergistic or superior to the pure modified cobratoxin product.

Studies were undertaken to identify cobra venoms with cobratoxin by Poly Acrylamide Gel Electrophoresis (PAGE) and ion exchange chromatography. Only N. kaouthia and N. naja had appreciable cobratoxin levels. All other venoms were devoid of cobratoxin including N. nivea, the original prototypic venom used by Sanders in the polio studies. Indeed, it was confirmed that there were N. kaouthia and N. naja venoms that demonstrated a distinct absence of cobratoxin due to geographical variation. Sanders also reportedly employed N. naja and N. haje venoms (N. haje has been split into N. haje and N. annulifera). It therefore suggested that cobratoxin was not the “principle” antiviral component in detoxified cobra venoms and its' presence was not required for effective antiviral activity.

To test this hypothesis N.n.atra venom (Chinese cobra) was modified and examined for antiviral effects. Atra venom is not a significant source of alpha-cobratoxin but is employed as a source of alpha-cobrotoxin, the short-chained neurotoxin. It proved to have significant antiviral effects (FIG. 1) as indicated in table II. However, cobrotoxin is present at relatively low levels in the venom. Consequently other components may be involved such as phospholipases or cardiotoxins. Other venoms were assayed for activity and are listed in table 1.

Fenard et al. (1999) proved that certain venom phospholipases could inhibit the replication of HIV in Peripheral Blood Mononuclear Cells (PBMCS). The highest activity was observed in those phospholipases isolated from snakes such as elapids, all of which were neurotoxic. One such phospholipase product was Nigexine isolated from N. nigricollis. The catalytically inactivated Viper phospholipase showed poor activity in the HIV assay though an active phospholipase activity was reported not to be involved in HIV inhibition. This might suggest that the preferred phospholipase for use in anti-HIV assays should be derived from cobras.

With the success of N. atra venom in the inhibition of HIV replication other cobra venoms that were known to possess little cobratoxin were tested for efficacy including: N. nigricollis, N. haje and N. nivea. N. nivea proved to be as effective as the N. naja product confirming the redundancy for cobratoxin as an active component. Generally, all cobra venom preparations were as effective at inhibiting HIV replication as was modified cobratoxin (FIG. 2). While Nigexine is usually purified from N. nigricollis venom it does not exclude its presence in other elapid venoms and a possible contribution to synergistic activity. Cobra venom is relatively abundant and the ability to utilize it as a crude raw material makes it simpler and cheaper to produce that any single venom component such as purified cobratoxin or Nigexine. It remains to be determined if the antiviral activity at least against HIV is associated with a single unknown venom component or a consequence of synergism between venom constituents. Indeed, cobratoxin's isolation as an antiviral component by Miller et al (1977) now appears entirely fortuitous.

### Production Techniques

Administration of a highly toxic substance such as cobratoxin for therapeutic purposes is fraught with obvious difficulties, even when highly diluted. As a diluted substance, its potential effectiveness is reduced, and due to its high affinity for the nAchR, continued use could result in accumulation of the toxin at neuromuscular junctions and the diaphragm with the potential for adverse events. Tu (1973) has indicated that the curaremimetic alpha-neurotoxins of cobra and krait venoms loose their toxicity upon either oxidation or upon reduction and alkylation of the disulfide bonds. The procedures used for detoxification described here are based upon the work of Sanders, who preferred the use of hydrogen peroxide (Sanders, et al., 1975). Loss of toxicity in oxidized venom can be determined by intraperitoneal (IP) injection of excess levels of the modified complex into mice. In general, injection of 4 mcg of natural cobra venom is lethal to a 25 g mouse. After detoxification, IP injection of a 5 mg modified cobra venom is non-toxic. This represents at least a 1250 fold reduction of toxicity.

In a preferred embodiment, the method of the present invention is used to prepare inactivated forms of elapid venoms, and more preferably venoms listed in the group below.

| | |
|---|---|
| Snake Venoms | Naja sp., Bungarus sp., Ophiophagus sp., |
| | Hemachatus sp., Boulengeria sp., Pseudohaje |
| | sp., Walterinnesia sp., Dendroaspis sp., Elaps |
| | sp., Acanthophis sp., Notechis sp., Oxyuranus |
| | sp., Pseudechis sp., Pseudonaja sp., Aipysurus |
| | sp., Astrotia sp., Enhydrina sp., Hydrophis sp., |
| | Lapemis sp., Laticauda sp., Pelamis sp., |

To inhibit infection of cells by HIV in vitro, cells are treated with the Modified venom (mCV) of the invention, either prior to, concurrently or following the addition of virus. Inhibition of infection of the cells by the mCV of the invention is assessed by measuring the replication of virus in the cells, by identifying the presence of viral nucleic acids and/or proteins in the cells, for example, by performing PCR, Southern, Northern or Western blotting analyses, reverse transcriptase (RT) assays, or by immunofluorescence or other viral protein detection procedures. The amount of mCV and virus to be added to the cells will be apparent to one skilled in the art from the teaching provided herein.

To inhibit infection of cells by HIV in vivo, the mCV of the invention, or a derivative thereof, is administered to a human subject who is either at risk of acquiring HIV infection, or who is already infected with HIV. Prior to administration, the mCV, or a derivative thereof, is suspended in a pharmaceutically acceptable formulation such as a saline solution or other physiologically acceptable solution which is suitable for the chosen route of administration and which will be readily apparent to those skilled in the art of mCV preparation and administration.

Typically, the mCV is administered in a range of 0.1 mcg to 2 mg of protein per dose. Approximately 1-10 doses are administered to the individual at intervals ranging from once per day to once every few years. The mCV may be administered by any number of routes including, but not limited to, subcutaneous, intramuscular, intravenous, intradermal, or intravaginal routes of administration. The mCV of the invention may be administered to the patient in a sustained release formulation using a biodegradable biocompatible polymer, or by on-site delivery using micelles, gels and liposomes, or rectally (e.g., by suppository or enema). The appropriate pharmaceutically acceptable carrier will be evident to those skilled in the art and will depend in large part upon the route of administration.

### EXAMPLES

### Example 1

Venom Modification

Venom from the Thailand cobra (Naja kaouthia) was purchased from Biotoxins (Florida) or Kentucky Reptile Zoo (Kentucky). Employing the procedure described by Sanders (U.S. Pat. No. 3,888,977) and Miller, et al. (1977) the reactive molecule, hydrogen peroxide, the precursor protein is modified through the addition of oxygen molecules.

Cobratoxin and other cobra venoms were detoxified in this manner.

### Example 2

Toxicity Assay in Mice

The endpoint of the above reactions are most easily determined by assessing the toxicity of the preparation in mice. Mice are sensitive to the actions of many venoms particularly to that of snakes. If the animal survives overnight it is accepted that the material is not lethal and defines the endpoint of the assay. By administering the composition of the invention at set periods a reduction in the material's toxicity can be observed as an increase in time to death. When 5 mg of the protein solution can be administered without inducing death then the reaction process is complete. It is at this point that the solution takes on its antiviral properties and native cobratoxin does not demonstrate antiviral activity in similar assays.

### Examples 3

Antiviral Experiments with Modified Venom.

Based upon findings that modified venoms and modified cobratoxin has antiviral properties in addition to an observed amino acid sequence homology between HIV-1 gp120 and cobratoxin, the ability of oxidized venoms to block in vitro HIV-1 infection in a thymus explant system and in PHA stimulated PBMC was examined. PHA stimulated PBMC were infected with a TCID₅₀ of 200 and 1000 of virus (R5 isolate HIV-1_{Bal} or X4 isolate HIV-1_{Lai}).

As a generalized procedure for the two laboratories involved in the in vitro testing of oxidized purified alpha neurotoxin and oxidized venom, the following was performed: PBMC from fresh, HIV-1 non-infected buffy coat cells obtained from healthy donors at local blood banks were purified by the Ficoll method. The buffy coat cells were maintained at room temperature until centrifugation. Purified PBMC were re-suspended at 1E6-3E6 cells/mL RPMI medium supplemented with 10% human AB serum and immediately treated with 5 ug PHA/mL suspension. Two to three days later, cells were counted and used for examination of infection. As a standard procedure, cells were incubated in propagation media, consisting of RPMI media supplemented with 10% human AB serum and 50 units IL2/mL, at a density of 6E6 cells per mL and incubated with 200-1000 TCID₅₀ HIV-1/mL×10E6 PBMC. Infection was allowed for 2 hours at 37° C. and the unbound virus was washed away by two washes with propagation media. 200,000 cells were suspended in 180 uL of propagation media and placed in 96 well plates (U bottom). Twenty uL of a 10× stock of the corresponding dilution of the drug was added to each well. Infections were performed in triplicate and controls containing 1 uM AZT were run in parallel as controls to confirm the validity of the assay. The cultures were incubated at 37° C. for 4 days. At that time, 90 uL of media was removed and replaced with 100 uL of propagation media containing the corresponding dilution of drug. The amount of p24 accumulated in the culture was estimated 3 days later (7 days post infection) with a Becton-Dickenson p24 ELISA. Routinely, a few samples were chosen and 10E-2 to 10E-4 dilutions of culture supernatant were prepared to estimate the linearity of the assay. The results from these experiments are summarized in Table II.

TABLE II
Summary of anti-HIV study results.

| Detoxified Product | IC50(mg/ml) |
|---|---|
| Assay 1 | |
| Naja naja (batch T10)* | 3 |
| Naja naja atra 1 | 14 |
| Naja naja atra 2 | 22 |
| Naja kaouthia | 200 |
| modified alpha-cobratoxin (batch T34)* | 34 |
| Assay 2 | |
| Naja naja (batch T10)* | 116 |
| Naja naja | 60 |
| Naja nivea | 18 |
| Naja nigricollis | >200 |
| Naja haje | 200 |
| modified alpha-cobratoxin (batch T34)* | >200 |

*Contrasting results arise from assay variability

What is claimed is:

1. A method of treatment of animals suffering from retroviral infections comprising administering to the animal a disease mitigating amount of a detoxified modified cobra venom composition.

2. The method of claim 1 wherein in humans a dosage of the composition ranges substantially from 0.00013 mg to 0.13 mg of the modified venom per Kg body weight.

3. The method of claim 5 wherein the dosage is from 0.2 to 2 ml.

4. The method of claim 5 wherein the dosage is administered substantially with a frequency of from every other week to daily.

5. The method of claim 5 wherein the dosage is administered at least weekly.

6. The method of claim 5 wherein the dosage is administered at least daily.

7. The method of claim 5 wherein the administration is by one of injection.

8. The method of claim 10 wherein administration by injection is by one of subcutaneous, intramuscular and intravenous.

9. The method of claim 1 which further comprises a viral condition benefitting from improved immunological functioning and reduced viral load.

10. The method of claim 1 wherein the retroviral infection is selected from a group comprising human immunodeficiency virus (HIV), simian immunodeficiency virus (SIV), feline immunodeficiency virus (FIV), bovine immunodeficiency virus (BIV) and equine acquired immunodeficiency virus (EAIV).
